(19) United States
(12) Patent Application Publication
(10) Pub. No.: US 2023/0405260 A1
(43) Pub. Date: Dec. 21, 2023

(54) Home Relaxation and Entertainment Device

(71) Applicant: Tracyann Marshall, Newark, NJ (US)

(72) Inventor: Tracyann Marshall, Newark, NJ (US)

(21) Appl. No.: 17/841,419

(22) Filed: Jun. 15, 2022

(51) Int. Cl. A61M 21/00; A61N 1/04; A61N 1/36

(52) U.S. Cl. CPC A61M 21/00; A61N 1/0492; A61N 1/36014; G06F 3/167

(57) ABSTRACT

The Home Relaxation Device gives people control over their home environment to relax and reduce stress. The Home Relaxation Device provides physical, mental, emotional and spiritual healing of the body by showing a projection and sounds from nature and music. The Home Relaxation Device is the size of an average living room wall, with a flat surface and thin edges. Automatic membership with purchase of a Home Relaxation Device will give access to the product with a one time sign-in code. No other account information would be shared or displayed to the actual product at home; instead, members can access their account information online or through the app. The Home Relaxation Device will be controlled with a remote, app and the user's voice.

### BACKGROUND

An electronic visual display, informally a screen, is a display device for presentation of images, text, or video transmitted electronically, without producing a permanent record. Electronic visual displays include television sets, computer monitors, and digital signage. By the above definition, an overhead projector (along with screen onto which the text, images, or video is projected) could reasonably be considered an electronic visual display since it is a display device for the presentation of an images, plain text, or video transmitted electronically without producing a permanent record. They are also ubiquitous in mobile computing applications like tablet computers, smartphones, and information appliances.

A mobile application, also referred to as a mobile app or simply an app, is a computer program or software application designed to run on a mobile device such as a phone, tablet, or watch. Mobile applications often stand in contrast to desktop applications which are designed to run on desktop computers, and web applications which run in mobile web browsers rather than directly on the mobile device.

### SUMMARY OF THE INVENTION

The Home Relaxation Device gives people control over their home environment to relax and reduce stress. The Home Relaxation Device provides physical, mental, emotional and spiritual healing of the body. After a long stressful day, the user can unwind and find peace with this invention. The parts of the device are environmentally-friendly, and no personal information is stored on the device itself. Automatic membership with purchase of a Home Relaxation Device will give access to the product with a one time sign-in code. No other account information would be shared or displayed to the actual product at home; instead, members can access their account information online or through the app. The app's remote access user profile features cautionary labels (such as “keep away from fire”), and operates best in dry environments. The device is easy to set-up. The Home Relaxation Device varies in size and shape. The device has a flat surface and thin edges on which to project a four-dimensional holographic live display of sounds and sensations from nature and music. The Home Relaxation Device will be controlled with a remote, app and the user's voice. Also, a DHDA Digital Holographic Data Augmentation includes an augmented reality display and a holographic reality display. The device further includes a flat surface and thin edges on which to project four or more dimensional holographic live augmentations. Future dimensions are implicitly embodied herein and reserved explicitly to the Applicant. The disclosed device is controlled with remote, App and touch but not limited thereto. The disclosed device will be coded to include an entire metaverse embodiment. Users are able to enjoy a different or alternate reality, world, environment and place. The disclosed device is accessed with a personalized pin code. The disclosed device uses time zones reflective of a user's time zone where use and operation occurs. Users have the option to use the disclosed device during the night or day. Default is a realistic time zone unless changed by the user.

A relaxation and entertainment system disclosed includes a plurality of sensors configured to monitor sense status for a user via a wireless telemetry thereof. The disclosure also includes a respective plurality of nerve conductors or stimulators disposed to increase or decrease a nerve response in reaction to the plurality of sensors to create relaxation. The disclosure further includes a feedback circuit configured to process the wireless telemetry and to drive the nerve conductors to create a steady state relaxation in the user.

### BRIEF DESCRIPTION OF THE DRAWINGS

FIG. 1 is a flow chart of the steps on how to use the system and app in accordance with an embodiment of the present disclosure.

FIG. 2 depicts an e-motion patch configured to create respective sensations on a user based on a feedback circuit via monitoring senses and heart activity in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

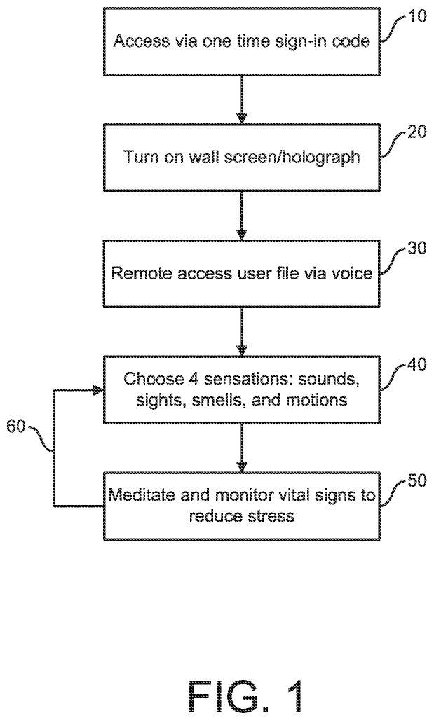

FIG. 1 is a flow chart of the steps on how to use the system and app in accordance with an embodiment of the present disclosure. Step 10 is to access the app via a one time sign-in code. Step 20 is to turn on the wall screen/holograph. Step 30 is remotely accessing the user file via voice commands. Step 40 is to choose four sensations out of sounds, sighs, smells, and motions. Step 50 is to meditate and monitor vital signs to reduce stress. A loop 60 from step 50 to step occurs via a feedback circuit designed to increase pleasurable sensations, sounds, sights, smells and motions when a user's vital signs are lagging a desired stasis such as a hypostatic condition. Based on a hyperstatic user monitoring, a nerve stimulation is decreased or at a predetermined frequency and amplitude cancels out the hyperactive stasis.

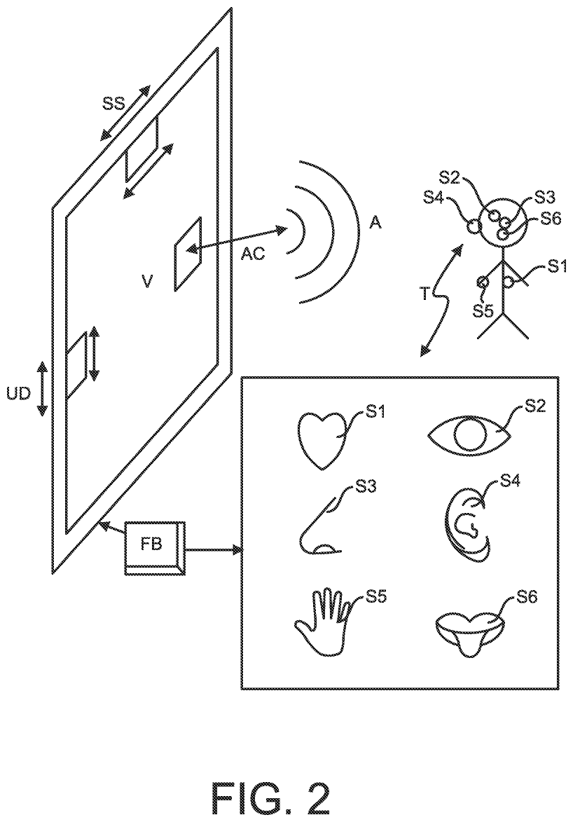

FIG. 2 depicts an e-motion patch configured to create respective sensations on a user based on a feedback circuit via monitoring senses and heart activity in accordance with an embodiment of the present disclosure. The depiction includes the stimulator V implemented as a large screen or as a small patch which is enabled to create move up and down, across and side to side sensations on respective parts of a user's respective nerve via a wireless telemetry input from a feedback circuit. The sound waves A are created by a vibration of the stimulator V at a predetermined frequency and amplitude. The respective sensors S1 for heart activity, S2 for visual activity, S3 for olfactory activity, S4 for aural activity, S5 for tactile activity and S6 for taste are wirelessly in communication with the patch P via the feedback circuit FB. The respective sensors monitor user activity and increase stimulation via an amplifier in the feedback circuit FB. The patch P is held adjacent a user's respective sense via an adhesive. Micromotors M actuate the respective up and down, side to side and across sensations. The telemetry T transmits information between the sensors and the stimulator V and the feedback FB circuits.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A relaxation system comprising:
a plurality of sensors configured to monitor sense status for a user via a wireless telemetry thereof;
a respective plurality of nerve stimulators disposed to increase or decrease a nerve response in reaction to the plurality of sensors; and
a feedback circuit configured to process the wireless telemetry and to drive the nerve stimulators to create a steady state relaxation in the user.

2. The system of claim 1, further comprising a remote one time access code for a user of the system.

3. The system of claim 1, further comprising a wall screen holograph configured to provide visual feedback to the user based on the plurality of sensors.

4. The system of claim 1, further comprising a remote access thereto via a user file voice activation.

5. The system of claim 1, wherein the feedback circuit is calibrated to a user's stress less baseline.

6. The system of claim 1, further comprising a feedback loop connecting the nerve stimulation and the sensor monitors.

7. The system of claim 1, wherein the plurality of nerve stimulators are configured to created a frequency of stimulation in excess of a user's recorded hypostasis.

8. The system of claim 1, wherein the plurality of nerve stimulators are configured to create a frequency of stimulation less than a user's recorded hyperstasis.

9. The system of claim 1, wherein the feedback circuit is disposed near the nerve stimulators.

10. The system of claim 1, further comprising a graphic user interface on a cell phone.

11. The system of claim 1, wherein one of the nerve stimulators is a large screen.

12. The system of claim 1, wherein one of the nerve stimulators is a small dermal patch.

13. The system of claim 1, wherein the feedback circuit is in electrical communication with the plurality of sensors and the respective plurality of nerve stimulators via a wireless transmission circuit.

14. The system of claim 1, further comprising a plurality of micro motors configured to create a three dimensional vibratory stimulation.

15. The system of claim 1, further comprising a nerve frequency and amplitude stimulation which cancels out a nerve hyperstasis frequency and amplitude to generate relaxation.

16. The system of claim 1, further comprising a nerve frequency and amplitude stimulation which cancels out a nerve hypstasis frequency and amplitude to generate relaxation.
